(19) United States
(12) Patent Application Publication
(10) Pub. No.: US 2010/0034798 A1
(43) Pub. Date: Feb. 11, 2010

(54) COMPOSITION AND METHOD OF USE FOR INTESTINAL HEALTH

(76) Inventor: David VanKomen, Draper, UT (US)

Correspondence Address: Workman Nydegger;1000 Eagle Gate Tower, 60 East South Temple, Salt Lake City, UT, 84111, US

(21) Appl. No.: 12/580,100

(22) Filed: Oct. 15, 2009

(51) Int. Cl. A61K 31/4415; A61P 1/00

(52) U.S. Cl. 424/94.5

(57) ABSTRACT

Therapeutic compositions can include ingredients to be useful in methods for improving or maintaining proper intestinal health. The therapeutic compositions can be used for maintaining a healthy intestinal tract by providing essential nutrients for intestinal function. Also, the therapeutic compositions can improve intestinal health by providing functional ingredients that can be useful for returning the intestinal tract to a healthy condition for optimal performance. The therapeutic compositions can be beneficial for small and large intestines, and other aspects of the GI tract as well as for the general heath of an individual.

### BACKGROUND

The digestive tract (i.e., GI tract) is the system of organs within multicellular animals that takes in food, digests it to extract energy and nutrients, and expels the remaining waste. The major functions of the GI tract are ingestion, digestion, absorption, and defecation. The GI tract differs substantially from animal to animal. Some animals have multi-chambered stomachs, while some animals' stomachs contain a single chamber. In a normal human adult male, the GI tract is approximately 6.5 meters (20 feet) long and consists of the upper and lower GI tracts.

The lower GI tract has important functions in the absorption of nutrients from consumed foods and beverages. The lower GI tract includes small intestine (e.g., duodenum, jejunum, and ileum), and the large intestine (e.g., cecum, colon, and rectum). However, when there are health problems associated with the GI tract, the overall health of an organism can be compromised. In part, the health problems may be attributed to a decreased function of the intestines, which may inhibit proper absorption of some or all types of nutrients from ingested food and beverages.

Poor intestinal health may account for the second greatest number of hospital admissions. In fact, four out of ten people visiting their doctors are there because of gastrointestinal complaints such as: diarrhea, excess gas, loss of weight, excess mucus, cramping, blood loss, severe constipation, and others. Not only are these disorders widespread, they exact a high toll in pain, suffering and expense. However, many conditions associated with poor intestinal health can be overcome by maintaining proper intestinal functions and conditions.

Therefore, there is a need for compositions and methods of administration that can provide improved intestinal health. Also, it would be advantageous to have a therapeutic regimen that improves or maintains proper intestinal health.

### BRIEF SUMMARY OF THE INVENTION

In one embodiment, the present invention can be a therapeutic composition for intestinal health. Such a composition can include: an antioxidant component; a vitamin B-3 component; a vitamin B-5 component; a vitamin B-6 component; a vitamin B-12 component; and a biotin component, wherein the composition has a therapeutically effective amount of the components to improve intestinal health. These ingredients can be present as follows: antioxidant component from about 5 mg to about 9 mg; vitamin B-3 component from about 40 mg to about 90 mg; vitamin B-5 component from about 40 mg to about 90 mg; vitamin B-6 component from about 40 mg to about 90 mg; vitamin B-12 component from about 4 mg to about 9 mg; and biotin component from about 40 mg to about 90 mg.

In one embodiment, the therapeutic composition can further include: a caffeine component; a high ORAC fruit component; a resveratrol component; a green tea component; or an acai component. These ingredients can be present as follows: caffeine component from about 50 mg to about 100 mg; high ORAC fruit component from about 30 mg to about 80 mg; resveratrol component from about 30 mg to about 80 mg; green tea component from about 30 mg to about 80 mg; or acai component from about 10 mg to about 60 mg.

In one embodiment, the therapeutic composition can further include: a pomegranate component; or a concord grape component. These ingredients can be present as follows: pomegranate component at about 500 mg to about 3 g; or concord grape component at about 100 mg to about 900 mg.

In one embodiment, the therapeutic composition can further include: a flavorant; a colorant; a malic acid component; a non-sugar sweetener component; a rosemary extract component; a chelator; a citric acid; or a preservative. These ingredients can be present as follows: flavorant at about 10 mg to about 90 mg; colorant at about 75 mg to about 500 mg; malic acid component at about 50 mg to about 100 mg; non-sugar sweetener component at about 5 mg to about 100 mg; rosemary extract component at about 1 mg to about 100 mg; chelator at about 0.5 mg to about 10 mg; citric acid at about 50 mg to about 100 mg; or preservative at about 10 mg to about 60 mg.

In one embodiment, the therapeutic composition can include one or more of the following: bromelain; a mangosteen component; a papaya component; a blueberry component; a ginger root component; a citrus bioflavinoid component; or a pro-biotic component.

In one embodiment, the therapeutic composition can be characterized as follows: a carrier from about 50% to about 95% by weight or volume; antioxidant component from about 0.01% to 0.02% by weight or volume; vitamin B-3 component from about 0.01% to 0.2% by weight or volume; vitamin B-5 component from about 0.01% to 0.2% by weight or volume; vitamin B-6 component from about 0.01% to 0.2% by weight or volume; vitamin B-12 component from about 0.001% to 0.02% by weight or volume; biotin component from about 0.101% to 0.19% by weight or volume; caffeine component from about 0.1% to 0.2% by weight or volume; high ORAC fruit component from about 0.01% to 0.2% by weight or volume; resveratrol component from about 0.01% to 0.2% by weight or volume; green tea component from at about 0.01% to 0.2% by weight or volume; acai component from about 0.01% to 0.2% by weight or volume; a pomegranate component from about 1% to 6% by weight or volume; a concord grape component about 0.5% to 2% by weight or volume; flavorant in an effective amount sufficient to provide a desired flavoring; colorant in an effective amount sufficient to provide a desired color; malic acid component from about 0.1% to 0.2% by weight or volume; non-sugar sweetener component from about 0.01% to 0.1% by weight or volume; rosemary extract component from about 0.005% to 0.1% by weight or volume; chelator from about 0.001% to 0.01% by weight or volume; citric acid from 0% to 0.2% by weight or volume; and preservative in an effective amount sufficient to preserve the composition.

In one embodiment, the therapeutic composition can be characterized as follows: a carrier; antioxidant component from about 5 mg to about 9 mg; vitamin B-3 component from about 40 mg to about 90 mg; vitamin B-5 component from about 40 mg to about 90 mg; vitamin B-6 component from about 40 mg to about 90 mg; vitamin B-12 component from about 4 mg to about 9 mg; biotin component from about 40 mg to about 90 mg; caffeine component from about 50 mg to about 100 mg; high ORAC fruit component from about 30 mg to about 80 mg; resveratrol component from about 30 mg to about 80 mg; green tea component from about 30 mg to about 80 mg; acai component from about 10 mg to about 60 mg; pomegranate component at about 500 mg to about 3 g; concord grape component at about 100 mg to about 900 mg; a flavorant at about 10 mg to about 90 mg; a colorant at about 75 mg to about 500 mg; a malic acid component at about 50 mg to about 100 mg; a non-sugar sweetener component at about 5 mg to about 100 mg; a rosemary extract component at about 1 mg to about 100 mg; a chelator at about 0.5 mg to about 10 mg; a citric acid at about 50 mg to about 100 mg; and a preservative at about 10 mg to about 60 mg.

In one embodiment, a therapeutic composition for intestinal health can include: bromelain; a mangosteen component; a papaya component; a blueberry component; a ginger root component; a citrus bioflavinoid component; and a pro-biotic component.

In one embodiment, the present invention can provide a method for improving intestinal health in a subject. Such a method can include: identifying a subject in need of improved intestinal health; and administering a therapeutically effective amount of a composition to the subject. The composition can be any therapeutic composition as described herein.

In one embodiment, the method can include identifying the subject to be in need of reduced weight, and administering a therapeutically effective amount of the composition so as to reduce weight of the subject.

In one embodiment, the method can include identifying the subject to be in need of reduced fatigue, and administering a therapeutically effective amount of the composition so as to reduce fatigue in the subject.

In one embodiment, the method can include identifying the subject to be in need of increased energy, and administering a therapeutically effective amount of the composition so as to increase energy in the subject.

In one embodiment, the method can include identifying the subject to be in need of reduced parasites in the GI tract, and administering a therapeutically effective amount of the composition so as to reduce the parasites in the GI tract of the subject.

In one embodiment, the method can include identifying the subject to be in need of reduced toxins in the GI tract, and administering a therapeutically effective amount of the composition so as to reduce the toxins in the GI tract of the subject.

In one embodiment, the method can include identifying the subject to have bloating, and administering a therapeutically effective amount of the composition so as to decrease bloating in the subject.

In one embodiment, the method can include identifying the subject to be in need of improved digestion, and administering a therapeutically effective amount of the composition so as to improve digestion in the subject.

In one embodiment, the method can include identifying the subject to have celiac disease, and administering a therapeutically effective amount of the composition so as to treat celiac disease in the subject.

In any of the foregoing embodiment, the identifying step can be omitted, and the composition can be administered in the effective amount to the subject so as to obtain the desired benefit.

These and other embodiments and features of the present invention will become more fully apparent from the following description and appended claims, or may be learned by the practice of the invention as set forth hereinafter.

### DETAILED DESCRIPTION

Generally, the present invention includes therapeutic compositions and methods of administration for improving or maintaining proper intestinal health. The therapeutic compositions can be used for maintaining a healthy intestinal tract by providing essential nutrients for intestinal function. Also, the therapeutic compositions can improve intestinal health by providing functional ingredients that can be useful for returning the intestinal tract to a healthy condition for optimal performance. The therapeutic compositions can be beneficial for small and large intestines, and other aspects of the GI tract as well as for the general heath of an individual.

The therapeutic composition can be used for improving health problems associated with the different portions (e.g., duodenum, jejunum, ileum) of the small intestine as well as maintaining a healthy small intestine. A healthy small intestine can facilitate chemical digestion of ingested foods and beverages by breaking down proteins and polypeptides into amino acids; degrading lipids into fatty acids and glycerols; and degrading carbohydrates into simple sugars or monosaccharides (e.g., glucose). Additionally, a healthy small intestine can facilitate nutrient absorption. The therapeutic composition can be administered to an individual with an inadequately functioning small intestine, or to maintain a properly functioning small intestine. The improved colon function can provide for proper chemical digestion to breakdown food and beverage components as well as proper absorption of ingested nutrients, including nutrients that are generated by chemical digestion.

The therapeutic composition can be used for improving health problems associated with the different portions of the large intestine as well as maintaining the health of the large intestine. A healthy large intestine can facilitate nutrient absorption and compaction of solid waste into feces. On aspect of a healthy large intestine is the maintenance of bacterial flora, and absorption of products formed by the bacterial flora. The therapeutic composition can be administered to an individual with an inadequately functioning large intestine so as to improve intestinal function. The improved large intestine function can provide for compaction and formation of solid waste, and improved absorption of nutrients from ingested foods and beverages as well as from the bacterial flora. In one aspect, improving health of the large intestine includes maintaining or providing an environment adequate for bacterial flora.

The therapeutic composition can be used for improving colon-related health problems in an individual as well as maintaining a healthy colon. Various health conditions are associated with an inadequately functioning colon. Some of these conditions result in reduced function in properly storing and/or passing waste, and in reclaiming water to maintain a proper water balance, as well as absorbing various nutrients, such as vitamins. The therapeutic composition can be administered to an individual with an inadequately functioning colon so as to improve colon function. The improved colon function can provide for proper storage and/or passage of waste, improved reclaiming water to provide a proper water balance, and to improve absorption of nutrients.

The therapeutic composition can be used for colon cleansing. As such, the therapeutic composition can help in facilitating removal of fecal waste and toxins from the intestinal tract, such as from the colon. Often, the colon or other aspects of the intestinal tract can be subject to accumulations of putrefied feces that line the walls of the large intestine, which is problematic from a functional standpoint. Such a buildup of fecal matter can inhibit the proper function of the large intestine because the cells and junctions on the surface of the large intestine are covered and thereby inhibited from functioning, and it forms a layer that inhibits nutritional diffusion across the intestinal wall. Also, such a buildup of fecal matter can harbor parasites and/or pathogenic gut flora (e.g., harmful bacteria). Additionally, the buildup of feces can cause intestinal distention, which in itself can cause headaches, fatigue, loss of appetite and irritability. Accordingly, accumulations of putrefied feces can cause various specific and nonspecific symptoms and general ill-health. Thus, the therapeutic composition can facilitate and/or enhance the functionality of the large intestine for fecal compression and movement out of the organism (e.g., defecation).

The therapeutic composition can be used for detoxification of the intestinal tract. This can included maintaining or enhancing the ability of the intestinal tract to remove toxins from the body of an organism. There are various modes of entry for toxins, such as by ingestion of foods and beverages that contain toxins, into the intestinal tract. For example, toxins can arise in the intestinal tract from harmful bacteria, overeating, consuming non-nutritional or junk foods as well as processed foods, or from eating foods that contain toxins. By enhancing or maintaining a functional intestine, the therapeutic composition can be beneficial in helping the intestinal tract remove toxins.

The therapeutic composition can be configured as a liquid, emulsion, dispersion, gel, paste, powder, bar, solid, or the like. The form of the therapeutic composition can be altered by altering the ingredients, such as by altering the carrier.

In one embodiment, the present invention can include a carrier, such as a pharmaceutically acceptable carrier, that can carry the active agents for administration. Often, water is used alone or with other carriers. Other carriers can depend on the form of the composition. For example, a liquid composition can include water as a carrier, and gels can further include thickening agents (e.g., polysaccharides, celluloses, etc.). Other forms of the therapeutic composition can be prepared by using traditional ingredients for the selected form.

When water is included as a carrier, it can be any format of liquid water. This can include distilled, reverse osmosis, purified, or other water products. For example, the water can be present at about 50% to 97% by weight or volume, in another aspect from about 60% to 96.5%, in another aspect from about 70% to 96%, in another aspect from about 80% to 95.5%, and in yet another aspect from about 90% to 95% by weight or volume. In another example, the water can be present in a serving (e.g., 60 mL liquid serving) from about 50 to 70 grams, in another aspect from about 55 to about 65 grams, and in another aspect about 57 to 58 grams, or about 57.5 grams.

In one embodiment, the therapeutic composition can include an antioxidant component. Any edible antioxidant can be used. Examples of suitable antioxidants can include ascorbic acid, glutathione, lipoic acid, uric acid, carotenes, alpha-tocopherol (e.g., vitamin E), ubiquinol (e.g., coenzyme Q) or the like as well as pharmaceutically acceptable salts, acids, or bases thereof.

In one embodiment, the antioxidant is ascorbic acid or a pharmaceutically acceptable salt thereof, such as an edible ascorbate. Vitamin C or L-ascorbic acid or salts thereof is an essential nutrient for humans, a large number of higher primate species, a small number of other mammalian species (notably guinea pigs and bats), a few species of birds, and some fish. The ascorbic acid may also be used in the D-ascorbic acid format. Ascorbate (an ion of ascorbic acid) is required for a range of essential metabolic reactions in all animals and plants. It is made internally by almost all organisms, humans being a notable exception. Deficiency in this vitamin causes scurvy in humans and is attributed to other adverse health conditions. In living organisms, ascorbate is an antioxidant, since it protects the body against oxidative stress, and is a cofactor in several vital enzymatic reactions. The oxidized form is L-dehydroascorbate, which can be used because it can be reduced back to the active L-ascorbate form in the body by enzymes and glutathione.

For example, the antioxidant component can be present at about 0.01% to 0.02% by weight or volume, in another aspect from about 0.011% to 0.019%, in another aspect from about 0.012% to 0.017%, in another aspect from about 0.0125% to 0.015%, and in yet another aspect from about 0.0130% by weight or volume. In another example, the antioxidant can be present in a serving (e.g., 60 mL liquid serving) from about 5 to 9 milligrams, in another aspect from about 6 to about 8.5 milligrams, and in another aspect about 7 to 8 milligrams, or about 7.75 milligrams.

In one embodiment, the therapeutic composition can include a niacin component (i.e., vitamin B-3 component). The niacin component can be niacin, vitamin B3, nicotinic acid, or precursors or derivatives thereof such as other forms that include the corresponding amide (e.g., nicotinamide or niacinamide), where the carboxyl group has been replaced by a carboxamide group (CONH₂), as well as more complex amides and a variety of esters. The terms niacin, nicotinamide, and vitamin B₃ are often used interchangeably to refer to any one of this family of molecules, since they have a common biochemical activity. In the body, niacin is converted to nicotinamide and then to NAD, NADH, NAD+, NADPH, and NADP, and thus, niacin can be replaced or supplemented with the same.

For example, the vitamin B-3 component can be present at about 0.01% to 0.2% by weight or volume, in another aspect from about 0.101% to 0.19%, in another aspect from about 0.102% to 0.17%, in another aspect from about 0.1025% to 0.15%, and in yet another aspect from about 0.1030% by weight or volume. In another example, the vitamin B-3 can be present in a serving (e.g., 60 mL liquid serving) from about 40 to 90 milligrams, in another aspect from about 50 to about 80 milligrams, and in another aspect about 55 to 70 milligrams, or about 60 to 65 milligrams.

In one embodiment, the therapeutic composition can include a cyanocobalamin component. The cyanocobalamin component can be cyanocobalamin, vitamin B-12, or precursors or derivatives thereof. Cyanocobalamin is a common vitamer of the B-12 vitamin family. It is the most famous vitamer of the family, because it is chemically the most air-stable, and it is the easiest to crystallize and therefore easiest to purify after it is produced by bacterial fermentation. The cyanide is added to the molecule by activated charcoal columns in purification. Thus, the use of this form of B-12 is the most wide-spread. In fact, vitamin B-12 is the name for a whole class of chemicals with B-12 activity, and cyanocobalamin is only one of these. As such, any vitamin B-12 can be used.

For example, the vitamin B-12 component can be present at about 0.001% to 0.02% by weight or volume, in another aspect from about 0.0101% to 0.019%, in another aspect from about 0.0102% to 0.017%, in another aspect from about 0.01025% to 0.015%, and in yet another aspect from about 0.0103% by weight or volume. In another example, the vitamin B-12 can be present in a serving (e.g., 60 mL liquid serving) from about 4 to 9 milligrams, in another aspect from about 5 to about 8 milligrams, and in another aspect about 5 to 7 milligrams, or about 6.0 to 6.5 milligrams.

A vitamer of a particular vitamin is any of the chemical compounds which exhibit vitamin activity. Very commonly “vitamins” are not single compounds, but rather each vitamin, which is defined by its biological activity, not its structure, is actually represented by a number of substances, all of which show vitamin activity. Typically, the vitamin activity of multiple vitamers is due to the body's (limited) ability to convert one vitamer to another, or many vitamers to the same enzymatic cofactor(s), which is active in the body as the important form of the vitamin. A set of chemicals may be (but is not always) grouped under an alphabetized vitamin “generic descriptor” title, such as “vitamin A,” which (for example) includes retinal, retinol, and many carotenoids. Examples of B-12 vitamers include cyanocobalamin, hydroxocobalamin, methylcobalamin, and 5-deoxyadenosylcobalamin (adenosylcobalamin-AdoB-12), which are all vitamers of B-12, and thus all possess “B-12 activity”. Another example is that both niacinamide and nicotinic acid (niacin) have vitamin B-3 activity.

In one embodiment, the therapeutic composition can include a B-5 vitamin component. An example of a B-5 vitamin is pantothenic acid or its salt pantothenate, such as calcium pantothenate in the D or L format, or precursors or derivatives thereof. Pantothenic acid is needed to form coenzyme-A (CoA), and is critical in the metabolism and synthesis of carbohydrates, proteins, and fats. In chemical structure, it is the amide between D-pantoate and beta-alanine. Pantothenate in the form of 4′phosphopantetheine is considered to be the more active form of the vitamin in the body.

For example, the vitamin B-5 component can be present at about 0.01% to 0.2% by weight or volume, in another aspect from about 0.101% to 0.19%, in another aspect from about 0.102% to 0.17%, in another aspect from about 0.1025% to 0.15%, and in yet another aspect from about 0.1030% by weight or volume. In another example, the vitamin B-5 can be present in a serving (e.g., 60 mL liquid serving) from about 40 to 90 milligrams, in another aspect from about 50 to about 80 milligrams, and in another aspect about 55 to 70 milligrams, or about 60 to 65 milligrams.

In one embodiment, the therapeutic composition can include a vitamin B-6 component. The vitamin B-6 component can include pyridoxine (e.g., pyridoxine HCL), pyridoxal, pyridoxamine, or precursors or derivatives thereof. Also, pyridozine can be converted to the biologically active form pyridoxal 5-phosphate, which can be used as a B-6 component. Pyridoxine assists in the balancing of sodium and potassium as well as promoting red blood cell production. It is linked to cardiovascular health by decreasing the formation of homocysteine. Lack of pyridoxine may cause anemia, nerve damage, seizures, skin problems, and sores in the mouth. It is also required for the production of the monoamine neurotransmitters serotonin, dopamine, norepinephrine and epinephrine, as it is the precursor to pyridoxal phosphate: cofactor for the enzyme aromatic amino acid decarboxylase.

For example, the vitamin B-6 component can be present at about 0.01% to 0.2% by weight or volume, in another aspect from about 0.101% to 0.19%, in another aspect from about 0.102% to 0.17%, in another aspect from about 0.1025% to 0.15%, and in yet another aspect from about 0.1030% by weight or volume. In another example, the vitamin B-6 can be present in a serving (e.g., 60 mL liquid serving) from about 40 to 90 milligrams, in another aspect from about 50 to about 80 milligrams, and in another aspect about 55 to 70 milligrams, or about 60 to 65 milligrams.

In one embodiment, the therapeutic composition can include a biotin component. Biotin, also known as vitamin H or B-7, is a water-soluble B-complex vitamin which is a cofactor in the metabolism of fatty acids and leucine, and it plays a role in gluconeogenesis. Biotin is necessary for cell growth, the production of fatty acids, and the metabolism of fats and amino acids. It plays a role in the citric acid cycle, which is the process by which biochemical energy is generated during aerobic respiration. Biotin not only assists in various metabolic reactions, but also helps to transfer carbon dioxide. Biotin is also helpful in maintaining a steady blood sugar level.

For example, the vitamin B-7 component can be present at about 0.01% to 0.2% by weight or volume, in another aspect from about 0.101% to 0.19%, in another aspect from about 0.102% to 0.17%, in another aspect from about 0.1025% to 0.15%, and in yet another aspect from about 0.1030% by weight or volume. In another example, the vitamin B-7 can be present in a serving (e.g., 60 mL liquid serving) from about 40 to 90 milligrams, in another aspect from about 50 to about 80 milligrams, and in another aspect about 55 to 70 milligrams, or about 60 to 65 milligrams.

In one embodiment, the therapeutic composition can include a caffeine component. Caffeine is part of the chemical mixtures and insoluble complexes guaranine found in guarana, mateine found in mate, and theine found in tea; all of which contain additional alkaloids such as the cardiac stimulants theophylline and theobromine, any of which can be used as a caffeine component. Also, caffeine has diuretic properties, which can be beneficial for colon cleansing.

For example, the caffeine component can be present at about 0.1% to 0.2% by weight or volume, in another aspect from about 0.11% to 0.19%, in another aspect from about 0.12% to 0.17%, in another aspect from about 0.125% to 0.15%, and in yet another aspect from about 0.135% by weight or volume. In another example, the caffeine component can be present in a serving (e.g., 60 mL liquid serving) from about 50 to 100 milligrams, in another aspect from about 60 to about 95 milligrams, and in another aspect about 70 to 90 milligrams, or about 80 to 85 milligrams.

In one embodiment, the therapeutic composition can include a fruit component having a high ORAC value (Oxygen Radical Absorbance Capacity). The fruit component can be a single fruit or a combination of fruits as well as concentrates and/or extracts thereof from any one or more of the following: wild blueberry, blueberry, cranberry, raspberry strawberry, prune, cherry, tart Cherry, bilberry, grape, seed extracts thereof, or the like. The ORAC value of 1 gram of the fruit component can be greater than about 1,000 ORAC, greater than about 1,500 ORAC, greater than about 2,500 ORAC, greater than about 3,000 ORAC, greater than about 4,000 ORAC, greater than about 5,000 ORAC, or greater than about 6,000 ORAC, or within any range between any of the recited ORAC values. Also, the fruit component can provide polyphenols, anthocyanidins, Vitamin C, and other organic acids that are naturally present in these fruits. In an example, the fruit component can include polyphenols, anthocyanins, proanthocyanidins, ellagic acid, chlorogenic acid, resveratrol, and quinic acid.

Another example is VitaBerry HiOrac Fruit Blend. VitaBerry™ Hi-ORAC Fruit Blend is a proprietary blend of fruit extracts & concentrated powders containing: Wild Blueberry (Vaccinium angustifolium) extract, Grape (Vitis vinifera) & Grape seed extract, Raspberry (Rubus idaeus) & Raspberry seed extract, Cranberry (Vaccinium macrocarpon), Prune (Prunus domestica), Tart Cherry (Prunus cerasus), Wild Bilberry (Vaccinium myrtillus) extract & Strawberry (Fragaria virginia)], Orange (Citrus sinensis). and Pomegranate (Punica granatum) min. It can be a 40% ellagic acid fruit extract.

For example, the high ORAC fruit component can be present at about 0.01% to 0.2% by weight or volume, in another aspect from about 0.011% to 0.19%, in another aspect from about 0.012% to 0.17%, in another aspect from about 0.0125% to 0.15%, and in yet another aspect from about 0.085% by weight or volume. In another example, the high ORAC fruit component can be present in a serving (e.g., 60 mL liquid serving) from about 30 to 80 milligrams, in another aspect from about 40 to about 75 milligrams, and in another aspect about 45 to 60 milligrams, or about 50 to 55 milligrams.

In one embodiment, the therapeutic composition can include a resveratrol component. Resveratrol (trans-resveratrol) is obtained from an extract from grape skin. Resveratrol is a phytoalexin produced naturally by several plants when under attack by pathogens such as bacteria or fungi. Resveratrol has also been produced by chemical synthesis and is sold as a nutritional supplement derived primarily from Japanese knotweed. In mouse and rat experiments, anti-cancer, anti-inflammatory, blood-sugar-lowering, and other beneficial cardiovascular effects of resveratrol have been reported. Resveratrol is found in the skin of red grapes and is a constituent of red wine. Experiments have shown that resveratrol treatment extended the life of fruit flies, nematode worms and short living fish. It aids with high cholesterol and high blood pressure, and helps with cell damage and reducing free radicals in the body.

For example, the resveratrol component can be present at about 0.01% to 0.2% by weight or volume, in another aspect from about 0.011% to 0.19%, in another aspect from about 0.012% to 0.17%, in another aspect from about 0.0125% to 0.15%, and in yet another aspect from about 0.085% by weight or volume. In another example, the resveratrol component can be present in a serving (e.g., 60 mL liquid serving) from about 30 to 80 milligrams, in another aspect from about 40 to about 75 milligrams, and in another aspect about 45 to 60 milligrams, or about 50 to 55 milligrams.

In one embodiment, the therapeutic composition can include green tea, such as in a green tea powder extract (Green Tea P.E.). Green tea is a type of tea made solely with the leaves of Camellia sinensis, which has undergone minimal oxidation during processing. Green tea contains polyphenols which are thought to improve health, particularly catechins, the most abundant of which is epigallocatechin gallate. In vitro and animal studies as well as preliminary observational and clinical studies of humans suggest that green tea can reduce the risk of cardiovascular disease and cancer as well as beneficially impact bone density, cognitive function, dental caries, and kidney stones. Green tea also contains carotenoids, tocopherols, ascorbic acid (vitamin C), minerals such as Cr, Mn, Se or Zn, and certain phytochemical compounds. It is a more potent antioxidant than black tea. It is also valued nutritionally for it high concentrations of Polyphenols and especially for its high concentration of EGCG (Epigallocatechin Gallate), an extremely potent antioxidant with profoundly beneficial effects on human health and that can scavenge and remove free radicals from the human tissues and organs. Recent scientific studies and research have demonstrated numerous health benefits from the oral supplementation of Green Tea P.E. and/or Epigallocatechin Gallate that may include protective effects from environmental stresses such as cigarette smoking and pollution. When taken in amounts not exceeding the recommended dose oral EGCG supplementation may also benefit individuals suffering from cancer, blood sugar swings, HPV virus infection, atherosclerosis, neurodegenerative disorders, as well as many other common maladies.

For example, the green tea component can be present at about 0.01% to 0.2% by weight or volume, in another aspect from about 0.011% to 0.19%, in another aspect from about 0.012% to 0.17%, in another aspect from about 0.0125% to 0.15%, and in yet another aspect from about 0.085% by weight or volume. In another example, the green tea component can be present in a serving (e.g., 60 mL liquid serving) from about 30 to 80 milligrams, in another aspect from about 40 to about 75 milligrams, and in another aspect about 45 to 60 milligrams, or about 50 to 55 milligrams.

In one embodiment, the therapeutic composition can include an acai component. Acai includes acai palm and aqai, which is one of several species of palm tree in the genus Euterpe cultivated for their fruit and superior hearts of palm. The species Euterpe oleracea is usually called Açaí Palm. Global demand for the fruit has expanded rapidly in recent years, and acai is now cultivated for that purpose primarily. The closely-related species Euterpe edulis (jucara) is now predominantly used for hearts of palm. The fruit, a small, round, black-purple drupe about 1 inch (25 mm) in diameter, similar in appearance and size to a grape but with less pulp, is produced in branched panicles of 700 to 900 fruits. Two crops of fruit are produced each year. The fruit has a single large seed about 0.25-0.40 inches (7-10 mm) in diameter. The acai component can be a product or extract from the fruit and/or seed.

For example, the acai component can be present at about 0.01% to 0.2% by weight or volume, in another aspect from about 0.011% to 0.19%, in another aspect from about 0.012% to 0.17%, in another aspect from about 0.0125% to 0.15%, and in yet another aspect from about 0.05% by weight or volume. In another example, the acai component can be present in a serving (e.g., 60 mL liquid serving) from about 10 to 60-milligrams, in another aspect from about 20 to about 50 milligrams, and in another aspect about 25 to 40 milligrams, or about 30 to 35 milligrams.

In one embodiment, the therapeutic composition can include a pomegranate component, which can be a product or extract from the fruit and/or seed. An example is pomegranate JC 65 Brix; however, the Brix value can vary. Pomegranate aril juice provides about 16% of an adult's daily vitamin C requirement per 100 ml serving, and is a good source of vitamin B-5 (pantothenic acid), potassium and antioxidant polyphenols. The most abundant polyphenols in pomegranate juice are the hydrolyzable tannins called punicalagins, which have free-radical scavenging properties in laboratory experiments. Punicalagins are absorbed into the human body and may have dietary value as antioxidants, but conclusive proof of efficacy in humans has not yet been shown. Pomegranate juice also includes phytochemicals such as beta-carotene, and polyphenols catechins, gallocatechins, and anthocyanins such as prodelphinidins, delphinidin, cyanidin, and pelargonidin. The ORAC of pomegranate juice is considered moderate when compared to other herbs and spices. The ORAC of pomegranate juice is measured at 2,860 units per 100 grams.

For example, the pomegranate component can be present at about 1% to 6% by weight or volume, in another aspect from about 2% to 5%, in another aspect from about 25% to 4%, and in yet another aspect about 3% by weight or volume. In another example, the pomegranate component can be present in a serving (e.g., 60 mL liquid serving) from about 500 milligrams to 3 grams, in another aspect from about 750 milligrams to about 2.5 grams, in another aspect about 1 gram to about 2 grams and in another aspect about 1.8 grams.

In one embodiment, the therapeutic composition can include a concord grape component, which can be a product or extract from the fruit and/or seed. An example is concord grape JC 68 Brix; however, the Brix value can vary.

For example, the concord grape component can be present at about 0.5% to 2% by weight or volume, in another aspect from about 0.75% to 1.9%, in another aspect from about 0.85% to 1.7%, in another aspect from about 0.95% to 1.5%, and in yet another aspect about 1% by weight or volume. In another example, the concord grape component can be present in a serving (e.g., 60 mL liquid serving) from about 100 to 900 milligrams, in another aspect from about 300 to about 800 milligrams, and in another aspect about 500 to 700 milligrams, or about 600 to 650 milligrams.

Degrees Brix is defined as soluble solids per 100 grams of juice (may not be exactly per 100 ml of juice) and is a measure of all soluble solids including pigments, acids, glycerol and sugar. Generally, the fermentable sugar content of grape juice is between 90 and 95% of the total soluble solids. The remainder can be classified as dry extract.

In one embodiment, the therapeutic composition can include a pomegranate flavoring component. An example is pomegranate flavor (103.27263). For example, the pomegranate flavoring component can be present at about 0.05% to 2% by weight or volume, in another aspect from about 0.075% to 1%, in another aspect from about 0.085% to 0.5%, in another aspect from about 0.095% to 0.25%, and in yet another aspect about 0.1% by weight or volume. In another example, the pomegranate flavoring component can be present in a serving (e.g., 60 mL liquid serving) from about 10 to 90 milligrams, in another aspect from about 30 to about 80 milligrams, and in another aspect about 50 to 70 milligrams, or about 60 to 65 milligrams. Also, other flavorants can be included within these ranges.

In one embodiment, the therapeutic composition can include one or more colorants. For example, the colorant can be an exberry shade bordeaux component, which is a natural colorant. Any edible colorants can be used. For example, the colorant component can be present at about 0.05% to 2% by weight or volume, in another aspect from about 0.075% to 1%, in another aspect from about 0.1% to 0.75%, in another aspect from about 0.2% to 0.5%, and in yet another aspect about 0.25% by weight or volume. In another example, the colorant component can be present in a serving (e.g., 60 mL liquid serving) from about 75 to 500 milligrams, in another aspect from about 100 to about 400 milligrams, and in another aspect about 125 to 300 milligrams, or about 150 to 175 milligrams.

In one embodiment, the therapeutic composition can include a malic acid component. Malic acid is an organic compound dicarboxylic acid and is the active ingredient in many sour or tart foods. The salts and esters of malic acid are known as malates. The malic acid component can be replace with or supplemented with related carboxylic acids or other compounds, such as: propionic acid; butyric acid; succinic acid; tartaric acid; crotonic acid; fumaric acid; pentanoic acid; butanol; butyraldehyde crotonaldehyde; sodium malate; or the like. For example, the malic acid component can be present at about 0.1% to 0.2% by weight or volume, in another aspect from about 0.11% to 0.19%, in another aspect from about 0.12% to 0.17%, in another aspect from about 0.125% to 0.15%, and in yet another aspect from about 0.135% by weight or volume. In another example, the malic acid can be present in a serving (e.g., 60 mL liquid serving) from about 50 to 100 milligrams, in another aspect from about 60 to about 95 milligrams, and in another aspect about 70 to 90 milligrams, or about 80 to 85 milligrams.

In one embodiment, the therapeutic composition can include a sugar and/or a sucralose for as a sweetener component as well as any non-sugar sweetener. Sugar (e.g., any form of sugar, such as fructose, sucrose, etc.) is a caloric sweetener, whereas sucralose is a zero-calorie sugar substitute artificial sweetener. Sucralose is approximately 600 times as sweet as sucrose (table sugar), twice as sweet as saccharin, and 3.3 times as sweet as aspartame. Unlike aspartame, it is stable under heat and over a broad range of pH conditions. Therefore, it can be used in baking or in products that require a longer shelf life. The commercial success of sucralose-based products stems from its favorable comparison to other low-calorie sweeteners in terms of taste, stability, and safety.

For example, the non-sugar sweetener component can be present at about 0.01% to 0.1% by weight or volume, in another aspect from about 0.02% to 0.09%, in another aspect from about 0.03% to 0.07%, in another aspect from about 0.04% to 0.06%, and in yet another aspect about 0.04% to about 0.05% by weight or volume. In another example, the non-sugar sweetener can be present in a serving (e.g., 60 mL liquid serving) from about 5 to 100 milligrams, in another aspect from about 10 to about 50 milligrams, and in another aspect about 15 to 35 milligrams, or about 25 to 30 milligrams. Sugars can be about 10 to 100 times the amount of non-sugar sweeteners

In one embodiment, the therapeutic composition can include a rosemary (also known as rosemary) extract component. An example is aquarox 10. Rosemary is extremely high in iron, calcium, and Vitamin B-6. For example, the rosemary component can be present at about 0.005% to 0.1% by weight or volume, in another aspect from about 0.01% to 0.09%, in another aspect from about 0.015% to 0.07%, in another aspect from about 0.02% to 0.06%, and in yet another aspect about 0.025% to about 0.05% by weight or volume. In another example, the rosemary can be present in a serving (e.g., 60 mL liquid serving) from about 1 to 100 milligrams, in another aspect from about 5 to about 50 milligrams, and in another aspect about 10 to 35 milligrams, or about 15 to 30 milligrams.

In one embodiment, the therapeutic composition can include a chelating agent. The chelating agent can be EDTA, EDDS, salts thereof, or the like. An example is sodium EDTA. For example, the chelating component can be present at about 0.001% to 0.01% by weight or volume, in another aspect from about 0.0015% to 0.009%, in another aspect from about 0.002% to 0.007%, in another aspect from about 0.0025% to 0.006%, and in yet another aspect about 0.003% to about 0.005% by weight or volume. In another example, the chelating can be present in a serving (e.g., 60 mL liquid serving) from about 0.5 to 10 milligrams, in another aspect from about 0.75 to about 5 milligrams, and in another aspect about 1 to 3 milligrams, or about 2 milligrams.

In one embodiment, the therapeutic composition can include a citric acid component. Citric acid is a weak organic acid, and it is a natural preservative and is also used to add an acidic, or sour, taste to foods and drinks. Citric acid can be used alone or in combination with malic acid and can be present in the same amounts as described herein, either alone, as a substitute, or in combination therewith.

In one embodiment, the therapeutic composition can include a preservative, such as sodium benzoate. For example, the preservative, such as sodium benzoate, component can be present at about 0.01% to 0.2% by weight or volume, in another aspect from about 0.011% to 0.19%, in another aspect from about 0.012% to 0.17%, in another aspect from about 0.0125% to 0.15%, and in yet another aspect from 0.06% by weight or volume. In another example, the acai component can be present in a serving (e.g., 60 mL liquid serving) from about 10 to 60 milligrams, in another aspect from about 20 to about 50 milligrams, and in another aspect about 30 to 40 milligrams, or about 35 to 38 milligrams.

In one embodiment, the therapeutic composition can include bromelain. Bromelain contains active substances that aid digestion and help reduce inflammation along with a variety of other health benefits.

In one embodiment, the therapeutic composition can include a mangosteen component, such as a product or extract. Mangosteen aids digestion and is full of rich xanthones and antioxidants, and contains fiber and vitamins to promote digestive health. Mangosteen also boosts energy and used as an herbal anti-depressant.

In one embodiment, the therapeutic composition can include a papaya component, such as a product or extract. Papaya is a digestive cleanser, and source of proteolytic enzymes, which help the body properly digest protein. It also boosts energy and contains essential vitamins.

In one embodiment, the therapeutic composition can include a blueberry component, such as a product or extract. The blueberry component can provide anti-aging properties, and has a high antioxidant activity.

In one embodiment, the therapeutic composition can include a ginger root component, such as a product or extract. The ginger root component is an antioxidant, and can help the digestive process by breaking down proteins, and can aid with high cholesterol and high blood pressure.

In one embodiment, the therapeutic composition can include a citrus bioflavinoids as products or extracts. Citrus bioflavinoids are antioxidants

In one embodiment, the therapeutic composition can include pro-biotic bacterium. The pro-biotics can help combat parasites, worms, and other harmful microorganisms in the intestinal tract, while boosting immune system. Usually, the probiotics are in a solid or powdered format.

In one embodiment, the therapeutic composition can include at least 4, 5, 6, 7, 8, 9, 10, 11, 12, 13, 14, 15, 16, 17, 18, 19, 20, 21, and/or 22 of the components listed above, which can be in any combination and in any relative amount with the proviso that the therapeutic composition can be used for the methods described herein.

In one embodiment, the therapeutic composition includes at least an antioxidant (e.g., vitamin C), a vitamin B-3, a vitamin B-5, a vitamin B-6, a vitamin B-12, and biotin. In another embodiment, the therapeutic composition includes at least an antioxidant (e.g., vitamin C), a vitamin B-3, a vitamin B-5, a vitamin B-6, a vitamin B-12, biotin, and a caffeine component. In another embodiment, the therapeutic composition includes at least an antioxidant (e.g., vitamin C), a vitamin B-3, a vitamin B-5, a vitamin B-6, a vitamin B-12, biotin, a caffeine component, and a fruit component with high ORAC value. In another embodiment, the therapeutic composition includes at least an antioxidant (e.g., vitamin C), a vitamin B-3, a vitamin B-5, a vitamin B-6, a vitamin B-12, biotin, a caffeine component, a fruit component with high ORAC value, and a resveratrol component. In another embodiment, the therapeutic composition includes at least an antioxidant (e.g., vitamin C), a vitamin B-3, a vitamin B-5, a vitamin B-6, a vitamin B-12, biotin, a caffeine component, a fruit component with high ORAC value, a resveratrol component, and a green tea component. In another embodiment, the therapeutic composition includes at least an antioxidant (e.g., vitamin C), a vitamin B-3, a vitamin B-5, a vitamin B-6, a vitamin B-12, biotin, a caffeine component, a fruit component with high ORAC value, a resveratrol component, a green tea component, and an acai component. In any of the foregoing embodiments, one or more of the antioxidant, vitamin B-3, vitamin B-5, vitamin B-6, and/or vitamin B-12 can be excluded so long as the functionality for the methods described herein is maintained. In any of the foregoing embodiments, one or more of the following can be included: a pomegranate component; a colorant; a malic acid component; a sucralose component (or other non-sugar sweetener); a rosemary extract component; a chelator; a citric acid component; a preservative; bromelain; a mangosteen component; a papaya component; a blueberry component; a ginger root component; a citrus bioflavinoid component; a pro-biotic component, combinations thereof, and the like. In one embodiment, the therapeutic composition includes: bromelain; a mangosteen component; a papaya component; a blueberry component; a ginger root component; a citrus bioflavinoid component; and a pro-biotic component.

In one embodiment, the therapeutic composition is a tablet or other solid dosage form, which can include powders, capsules, or the like. The dosage form can be administered 1, 2, 3, or 4 times a day depending on the format. In another example, a serving size can be 60 ml as a liquid, or from about 20 ml to about 90 ml, in another aspect from about 40 ml to about 80 ml, and in another aspect from about 50 ml to about 70 ml. Typically, a bottle of the therapeutic composition in a liquid format can be about 480 ml; however, other amounts can be provided. Each serving can provide the amount of each therapeutic component as described above. Also, tablets or other solid dosages can be configured and formulated based on the amounts, dosages, and servings sizes described herein. A preferred dosage can be a 60 ml shot of liquid in accordance with the foregoing, that is taken once or twice a day, which can include about 61 grams with about 52 grams being the carrier. When a solid, the amounts of components per dosage can be configured into one or more solid dosage forms for administration, such as one or more pills.

In one embodiment, the therapeutic composition can be used in a therapy for a person having intestinal problems. As such, the therapeutic composition can be provided/administered to person with intestinal problems so as to correct some intestinal problem.

The therapeutic composition can be used to increase or maintain colon and intestinal health, maintain & support healthy weight-loss, help reduce weight in subjects that are overweight, reduce fatigue, increase energy, combat parasites and worms, and help the removal toxins or toxin producers. Generally, the therapeutic composition can increase the overall health and performance of a body, and can be used to treat, inhibit, and or prevent bloating, digestion problems, celiac Disease, and any other intestinal and digestion problems, intestinal gas; acites; constipation; fatigue (chronic or acute); stress; or the like.

### EXAMPLES

### Example 1

A therapeutic composition in accordance with the present invention was prepared as shown in Table 1 below.

TABLE 1

| | Per serving | | |
|---|---|---|---|
| | of 60 ml | Per Bottle of | |
| Ingredients | (g) | 480 ml (g) | % |
| Reverse Osmosis (RO) Water | 57.3516582 | 458.1767232 | 94.508% |
| Vitamin C (as Ascorbic acid) | 0.007878 | 0.063024 | 0.013% |
| Niacin B3 | 0.062418 | 0.499344 | 0.103% |
| Cyanocobalamin B12 | 0.0062418 | 0.0499344 | 0.010% |
| Calcium D-Pantothenate | 0.062418 | 0.499344 | 0.103% |
| Pyridoxine HCL | 0.062418 | 0.499344 | 0.103% |
| Biotin | 0.062418 | 0.499344 | 0.103% |
| Caffeine Anhydrous | 0.08181 | 0.65448 | 0.135% |
| VitaBerry HiOrac FruitBlend | 0.05151 | 0.41208 | 0.085% |
| N81 | | | |
| Resveratrol (Grape Skin Extract) | 0.05151 | 0.41208 | 0.085% |
| Green Tea PE | 0.05151 | 0.41208 | 0.085% |
| Acai FPD | 0.0303 | 0.2424 | 0.050% |
| Pomegranate JC 65 Brix | 1.818 | 14.544 | 3.000% |
| Concord Grape JC 68 Brix | 0.606 | 4.848 | 1.000% |
| Pomegranate Flavor 103.27263 | 0.0606 | 0.4848 | 0.100% |
| Exberry Shade Bordeaux | 0.1515 | 1.212 | 0.250% |
| Malic Acid | 0.08181 | 0.65448 | 0.135% |
| Sucaralose | 0.026058 | 0.208464 | 0.043% |
| Aquarox 10 (Rosmary Extract) | 0.01515 | 0.1212 | 0.025% |
| Sodium EDTA | 0.0019998 | 0.0159984 | 0.003% |
| Sodium benzoate | 0.03636 | 0.29088 | 0.060% |

### Example 2

Subject A (male) indicated ill health for 2.5 years with a later diagnosis of celiac disease and a weight loss of about 32 lbs. After consuming a therapeutic composition in accordance with the present invention for only a limited time, Subject A reported an improvement in health, normal weight, and reduced symptoms attributed to celiac.

Subject B (male) consumed a therapeutic composition and indicated a feeling of generally improved health with decreased bloating and increased energy.

Subject C (male) had severe digestion and constipation issues, and consumed a therapeutic composition for three weeks. A report of digestion being improved dramatically and regular bowel movements related to the therapeutic composition and dosing regimen was provided.

Subjects D and E (both female) contracted extreme abdominal pain in Mexico that was not treatable by antibiotics. The subjects consumed a therapeutic composition for three weeks, and reported less ill health as more of the therapeutic dosage was consumed. After three weeks, both subjects were feeling normal and free of the abdominal pain.

Subject F (male) was overweight and had been unsuccessful in losing weight from diet and exercise. The subject consumed a therapeutic composition for 3 weeks and lost weight (e.g., 10 pounds) in that time.

Subject G (female) reported feeling extremely bloated and discomfort after meals that, and was not treatable by digestive enzymes. The subject consumed a therapeutic composition for two weeks and reported feeling better without the bloated or discomfort.

Subject H (male) had stomach problems due to being prescribed antibiotics for a significant period of time. After consuming a therapeutic composition for 3 weeks, the stomach problems were significantly reduced.

Subject I (female) was overweight, bloated, and chronically fatigued. After consuming the therapeutic composition for 2 months, the subject lost around 20 pounds and had reduced bloating as well as increased energy.

Subject J (male) reported being bloated and chronically fatigued. After consuming a therapeutic composition, the subject reported feeling less bloated with a dramatic increase in energy.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference in their entirety.

1. A therapeutic composition for intestinal health comprising:
an antioxidant component;
a vitamin B-3 component;
a vitamin B-5 component;
a vitamin B-6 component;
a vitamin B-12 component; and
a biotin component, wherein the composition has a therapeutically effective amount of the components to improve intestinal health.

2. A therapeutic composition as in claim 1, a serving of the therapeutic composition being characterized by the following:
antioxidant component from about 5 mg to about 9 mg;
vitamin B-3 component from about 40 mg to about 90 mg;
vitamin B-5 component from about 40 mg to about 90 mg;
vitamin B-6 component from about 40 mg to about 90 mg;
vitamin B-12 component from about 4 mg to about 9 mg; and
biotin component from about 40 mg to about 90 mg.

3. A therapeutic composition as in claim 1, further comprising one or more of the following:
a caffeine component;
a high ORAC fruit component;
a resveratrol component;
a green tea component; or
an acai component.

4. A therapeutic composition as in claim 2, a serving of the therapeutic composition being characterized by the following:
caffeine component from about 50 mg to about 100 mg;
high ORAC fruit component from about 30 mg to about 80 mg;
resveratrol component from about 30 mg to about 80 mg;
green tea component from about 30 mg to about 80 mg; or
acai component from about 10 mg to about 60 mg.

5. A therapeutic composition as in claim 3, further comprising one or more of the following:
a pomegranate component; or
a concord grape component.

6. A therapeutic composition as in claim 4, a serving of the therapeutic composition being characterized by the following:
pomegranate component at about 500 mg to about 3 g; or
concord grape component at about 100 mg to about 900 mg.

7. A therapeutic composition as in claim 5, further comprising one or more of the following:
a flavorant;
a colorant;
a malic acid component;
a non-sugar sweetener component;
a rosemary extract component;
a chelator;
a citric acid; or
a preservative.

8. A therapeutic composition as in claim 6, a serving of the therapeutic composition being characterized by the following:
a flavorant at about 10 mg to about 90 mg;
a colorant at about 75 mg to about 500 mg;
a malic acid component at about 50 mg to about 100 mg;
a non-sugar sweetener component at about 5 mg to about 100 mg;
a rosemary extract component at about 1 mg to about 100 mg;
a chelator at about 0.5 mg to about 10 mg;
a citric acid at about 50 mg to about 100 mg; or
a preservative at about 10 mg to about 60 mg.

9. A therapeutic composition as in claim 1, further comprising one or more of the following:
bromelain;
a mangosteen component;
a papaya component;
a blueberry component;
a ginger root component;
a citrus bioflavinoid component; or
a pro-biotic component.

10. A therapeutic composition as in claim 1, further comprising:
a caffeine component;
a high ORAC fruit component;
a resveratrol component;
a green tea component; and
an acai component.

11. A therapeutic composition as in claim 10, further comprising:
a pomegranate component; and
a concord grape component.

12. A therapeutic composition as in claim 11, further comprising:
a carrier;
a flavorant;
a colorant;
a malic acid component;
a non-sugar sweetener component;
a rosemary extract component;
a chelator;
a citric acid; and
a preservative.

13. A therapeutic composition as in claim 12, characterized by the following:
carrier from about 50% to about 95% by weight or volume;
antioxidant component from about 0.01% to 0.02% by weight or volume;
vitamin B-3 component from about 0.01% to 0.2% by weight or volume;
vitamin B-5 component from about 0.01% to 0.2% by weight or volume;
vitamin B-6 component from about 0.01% to 0.2% by weight or volume;
vitamin B-12 component from about 0.001% to 0.02% by weight or volume;
biotin component from about 0.101% to 0.19% by weight or volume;
caffeine component from about 0.1% to 0.2% by weight or volume;
high ORAC fruit component from about 0.01% to 0.2% by weight or volume;
resveratrol component from about 0.01% to 0.2% by weight or volume;
green tea component from at about 0.01% to 0.2% by weight or volume;
acai component from about 0.01% to 0.2% by weight or volume;
a pomegranate component from about 1% to 6% by weight or volume;
a concord grape component about 0.5% to 2% by weight or volume;
flavorant in an effective amount sufficient to provide a desired flavoring;
colorant in an effective amount sufficient to provide a desired color;
malic acid component from about 0.1% to 0.2% by weight or volume;
non-sugar sweetener component from about 0.01% to 0.1% by weight or volume;
rosemary extract component from about 0.005% to 0.1% by weight or volume;
chelator from about 0.001% to 0.01% by weight or volume;
citric acid from 0% to 0.2% by weight or volume; and
preservative in an effective amount sufficient to preserve the composition.

14. A therapeutic composition as in claim 12, characterized by the following:
antioxidant component from about 5 mg to about 9 mg;
vitamin B-3 component from about 40 mg to about 90 mg;
vitamin B-5 component from about 40 mg to about 90 mg;
vitamin B-6 component from about 40 mg to about 90 mg;
vitamin B-12 component from about 4 mg to about 9 mg;
biotin component from about 40 mg to about 90 mg;
caffeine component from about 50 mg to about 100 mg;
high ORAC fruit component from about 30 mg to about 80 mg;
resveratrol component from about 30 mg to about 80 mg;
green tea component from about 30 mg to about 80 mg;
acai component from about 10 mg to about 60 mg;
pomegranate component at about 500 mg to about 3 g;
concord grape component at about 100 mg to about 900 mg;
a flavorant at about 10 mg to about 90 mg;
a colorant at about 75 mg to about 500 mg;
a malic acid component at about 50 mg to about 100 mg;
a non-sugar sweetener component at about 5 mg to about 100 mg;
a rosemary extract component at about 1 mg to about 100 mg;
a chelator at about 0.5 mg to about 10 mg;
a citric acid at about 50 mg to about 100 mg; and
a preservative at about 10 mg to about 60 mg.

15. A therapeutic composition for intestinal health comprising:
bromelain;
a mangosteen component;
a papaya component;
a blueberry component;
a ginger root component;
a citrus bioflavinoid component; and
a pro-biotic component.

16. A method for improving intestinal health in a subject, the method comprising:
identifying a subject in need of improved intestinal health; and
administering a therapeutically effective amount of a composition to the subject, the composition comprising:
an antioxidant component;
a vitamin B-3 component;
a vitamin B-5 component;
a vitamin B-6 component;
a vitamin B-12 component; and
a biotin component, wherein the composition has a therapeutically effective amount of the components to improve intestinal health.

17. A method as in claim 16, the composition further comprising one or more of the following:
a caffeine component;
a high ORAC fruit component;
a resveratrol component;
a green tea component; or
an acai component.

18. A method as in claim 17, the composition further comprising one or more of the following:
a pomegranate component; or
a concord grape component.

19. A method as in claim 18, the composition further comprising one or more of the following:
a flavorant;
a colorant;
a malic acid component;
a non-sugar sweetener component;
a rosemary extract component;
a chelator;
a citric acid; or
a preservative.

20. A method as in claim 19, the composition being characterized by the following:
a carrier from about 50% to about 95% by weight or volume;
antioxidant component from about 0.01% to 0.02% by weight or volume;
vitamin B-3 component from about 0.01% to 0.2% by weight or volume;
vitamin B-5 component from about 0.01% to 0.2% by weight or volume;
vitamin B-6 component from about 0.01% to 0.2% by weight or volume;
vitamin B-12 component from about 0.001% to 0.02% by weight or volume;
biotin component from about 0.101% to 0.19% by weight or volume;
caffeine component from about 0.1% to 0.2% by weight or volume;
high ORAC fruit component from about 0.01% to 0.2% by weight or volume;
resveratrol component from about 0.01% to 0.2% by weight or volume;
green tea component from at about 0.01% to 0.2% by weight or volume;
acai component from about 0.01% to 0.2% by weight or volume;
a pomegranate component from about 1% to 6% by weight or volume;
a concord grape component about 0.5% to 2% by weight or volume;
flavorant in an effective amount sufficient to provide a desired flavoring;
colorant in an effective amount sufficient to provide a desired color;
malic acid component from about 0.1% to 0.2% by weight or volume;
non-sugar sweetener component from about 0.01% to 0.1% by weight or volume;
rosemary extract component from about 0.005% to 0.1% by weight or volume;
chelator from about 0.001% to 0.01% by weight or volume;
citric acid from 0% to 0.2% by weight or volume; and
preservative in an effective amount sufficient to preserve the composition.

21. A method as in claim 19, a serving of the composition being characterized by the following:
antioxidant component from about 5 mg to about 9 mg;
vitamin B-3 component from about 40 mg to about 90 mg;
vitamin B-5 component from about 40 mg to about 90 mg;
vitamin B-6 component from about 40 mg to about 90 mg;
vitamin B-12 component from about 4 mg to about 9 mg;
biotin component from about 40 mg to about 90 mg;
caffeine component from about 50 mg to about 100 mg;
high ORAC fruit component from about 30 mg to about 80 mg;
resveratrol component from about 30 mg to about 80 mg;
green tea component from about 30 mg to about 80 mg;
acai component from about 10 mg to about 60 mg;
pomegranate component at about 500 mg to about 3 g;
concord grape component at about 100 mg to about 900 mg;
a flavorant at about 10 mg to about 90 mg;
a colorant at about 75 mg to about 500 mg;
a malic acid component at about 50 mg to about 100 mg;
a non-sugar sweetener component at about 5 mg to about 100 mg;
a rosemary extract component at about 1 mg to about 100 mg;
a chelator at about 0.5 mg to about 10 mg;
a citric acid at about 50 mg to about 100 mg; and
a preservative at about 10 mg to about 60 mg.

22. A method as in claim 21, the method further comprising:
identifying the subject to be in need of reduced weight; and
administering a therapeutically effective amount of the composition so as to reduce weight of the subject.

23. A method as in claim 21, the method further comprising:
identifying the subject to be in need of reduced fatigue; and
administering a therapeutically effective amount of the composition so as to reduce fatigue in the subject.

24. A method as in claim 21, the method further comprising:
identifying the subject to be in need of increased energy; and
administering a therapeutically effective amount of the composition so as to increase energy in the subject.

25. A method as in claim 21, the method further comprising:
identifying the subject to be in need of reduced parasites in the GI tract; and
administering a therapeutically effective amount of the composition so as to reduce the parasites in the GI tract of the subject.

26. A method as in claim 21, the method further comprising:
identifying the subject to be in need of reduced toxins in the GI tract; and
administering a therapeutically effective amount of the composition so as to reduce the toxins in the GI tract of the subject.

27. A method as in claim 21, the method further comprising:
identifying the subject to have bloating; and
administering a therapeutically effective amount of the composition so as to decrease bloating in the subject.

28. A method as in claim 21, the method further comprising:
identifying the subject to be in need of improved digestion; and
administering a therapeutically effective amount of the composition so as to improve digestion in the subject.

29. A method as in claim 21, the method further comprising:
identifying the subject to have celiac disease; and
administering a therapeutically effective amount of the composition so as to treat celiac disease in the subject.

30. A method as in claim 29, the method further comprising:
administering the serving twice a day.
